(19) United States
(12) Patent Application Publication
(10) Pub. No.: US 2021/0353658 A1
(43) Pub. Date: Nov. 18, 2021

(54) COMPOSITION AND METHOD OF INHIBITING CORTISONE REDUCTASE

(71) Applicant: Amorepacific Corporation, Seoul (KR)

(72) Inventors: Eun Jeong CHOI, Yongin-si (KR); Euidong SON, Yongin-si (KR); Young Gyu KANG, Yongin-si (KR); Hyoung June KIM, Yongin-si (KR); Tae Ryong LEE, Yongin-si (KR)

(73) Assignee: Amorepacific Corporation, Seoul (KR)

(21) Appl. No.: 17/284,252

(22) PCT Filed: Oct. 10, 2019
(86) PCT No.: PCT/KR2019/013308

§ 371 Date: Apr. 9, 2021

(30) Foreign Application Priority Data
Oct. 11, 2018 (KR) 10-2018-0121283

(51) Int. Cl. A61K 31/7008; A61K 8/60; A61Q 19/00

(52) U.S. Cl. CPC A61K 31/7008; A61K 2800/10; A61Q 19/00; A61K 8/60

(57) ABSTRACT

Provided is a composition for inhibiting cortisone reductase, including a compound represented by a specific chemical formula as an active ingredient.

### CROSS REFERENCE TO RELATED APPLICATION

This application is a National Phase Patent Application and claims priority of International Application Number PCT/KR2019/013308, filed Oct. 10, 2019 which claims priority to and the benefit of Korean Patent Application No. 10-2018-0121283 filed in the Korean Intellectual Property Office on Oct. 11, 2018, the entire contents of each is incorporated herein by reference.

### TECHNICAL FIELD

This disclosure relates to a composition and a method of inhibiting cortisone reductase by applying it to the skin.

### BACKGROUND ART

Stress has been called a root of all illnesses since ancient times, and particularly in modern society, stress has excessively occurred because of various reasons such as social factors of study, work, marriage, parenting, and the like, and environmental factors such as weather, traffic, and the like for anyone regardless of sex or age, so it is recognized as a very serious social problem.

As our society has rapidly developed and diversified, roles required of modern people are increased, so that people suffering generalized anxiety disorders and mental disorders caused by many types of stress have increased. According to “A Study on Epidemiology of Mental Illness in 2006” published by the Ministry of Health and Welfare, “a one-year prevalence of mental illness” which refers to a percentage of people that experienced at least one type of mental illness for the year of 2006 was found to be 17.1%. This is around one person per 6 adults from greater than or equal to 18 years old to less than or equal to 64 years old, and ‘a lifetime prevalence of metal illness’ which is a percentage of people who experienced at least one type of mental illness for a whole life at the moment in 2006 was found to be 30%, which is one person per 3 adults. Considering the tendency toward increasing mental illness of adolescents caused by excessive academic enthusiasm or many types of stress, the prevalence for the entire population may be considered higher than the above.

Nowadays, anxiety disorder is treated by drug treatment along with a long-term psychotherapy in a clinic, and in the case of drug treatment, benzodiazepine-based anti-anxiety drugs such as diazepam, lorazepam, clonazepam, and alprazolam are mainly used, and azapirone-based buspirone is used as a drug for selectively acting on a serotonin receptor to selectively mitigate anxiety symptoms.

In addition, recently, researches on stress controlling materials derived from natural materials capable of compensating side effects of these drugs have been actively performed.

The present inventors continuously researched substances derived from natural products capable of preventing or treating mental stress-related diseases and have confirmed that in a mental stress situation, 11β-hydroxysteroid dehydrogenase type 1, cortisone reductase of keratinocytes present in the epidermis, is activated, increasing a concentration of cortisol in the epidermis (increasing a reduction degree from cortisone to cortisol), and in addition, skin barrier function deterioration phenomenon due to the activation of the 11β-hydroxysteroid dehydrogenase type 1 shows a completely different mechanism from that caused by other causes (e.g. physical injury or aging, etc.) not by the mental stress. Furthermore, it has been confirmed that specific compound extracted from soybean specifically inhibits the activity of the 11β-hydroxysteroid dehydrogenase type 1, thereby completing one aspect of the present disclosure.

On the other hand, Korean Patent Laid-Open Publication No. 2001-0011241 discloses a composition for skin protection having the same lipid composition and structural properties as an intercellular biological membrane existing between human skin cells but does not only focuses on a method of preparing a composition based on ceramide, cholesterol, and fatty acids, but also the composition includes simple ceramide that is difficult to apply to cosmetics due to the solubility problem and has no liquid crystal-type emulsified particles, and accordingly, there are problems of deteriorating stability and moisturizing power of a formulation and also deteriorating stability of the formulation after the preparation.

In addition, the composition is prepared by simply mixing similar substances to the skin lipid composition and thus has a limit in terms of skin-penetrating ability and skin moisturizing power.

Furthermore, up to now as well as in the conventional inventions including the aforementioned prior art, there is nothing known about substances capable of preventing or treating the skin barrier function deterioration by inhibiting the activation of cortisone reductase (11β-hydroxysteroid dehydrogenase 1) caused by the mental stress, not by physical injury or aging.

### DISCLOSURE

### Technical Problem

In an embodiment, a (cosmetic) composition capable of shortening the time of recovering a barrier function of the stratum corneum is provided by inhibiting the activity of 11β-hydroxysteroid dehydrogenase type 1, which is a factor in reducing skin barrier function due to mental stress and thus, by reducing the concentration of cortisol caused by mental stress.

### Technical Solution

According to an embodiment, a composition for inhibiting cortisone reductase includes a compound represented by Chemical Formula 1 as an active ingredient.

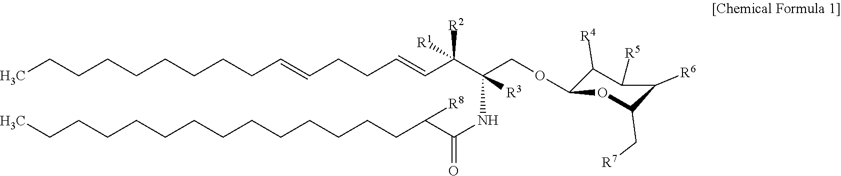

In Chemical Formula 1,

R¹ to R⁸ are each independently a hydrogen atom or a hydroxy group, provided that at least one of R¹ to R⁸ is necessarily a hydroxy group.

In Chemical Formula 1, R¹ and R³ may each independently be a hydrogen atom, and R² and R⁴ to R⁸ may each independently be a hydroxy group.

The compound represented by Chemical Formula 1 may be a soybean extract.

The cortisone reductase may be 11β-hydroxysteroid dehydrogenase type 1.

The compound represented by Chemical Formula 1 may be included in a concentration range of 0.01 μg/ml to 1,000 μg/ml.

The composition may be a cosmetic composition.

According to another embodiment, provided is a method of inhibiting cortisone reductase by applying a composition including an effective amount of the compound represented by Chemical Formula 1 as an active ingredient, to the skin.

### Advantageous Effects

According to an embodiment, the time of recovering a barrier function of the stratum corneum may be shortened by inhibiting the activity of 11β-hydroxysteroid dehydrogenase type 1, which is a factor in decreasing skin barrier function due to mental stress, and thus by reducing the concentration of cortisol caused by mental stress. In other words, it may specifically prevent and treat deterioration of a skin barrier function caused by mental stress which is one of the various reasons of deteriorating a skin barrier function.

### DESCRIPTION OF THE DRAWINGS

FIG. 1 is a result of measuring the concentration of cortisol in a cell culture solution using an ELISA method.

FIG. 2 is an RT-qPCR result for confirming the gene expression of keratin 10, which is a keratinocyte differentiation marker.

FIG. 3 is an RT-qPCR result for confirming the gene expression of keratin 1 which is a keratinocyte differentiation marker.

### MODES

Hereinafter, embodiments of one aspect of the present disclosure are described in detail so that those of ordinary skill in the art can easily implement one aspect of the present disclosure. However, one aspect of the present disclosure may be embodied in many different forms, and is not to be construed as limited to the example embodiments set forth herein.

In the present specification, the improvement of skin barrier function means reducing a concentration of cortisol by inhibiting the activity of 11β-hydroxysteroid dehydrogenase type 1 present in the damaged skin area due to mental stress, which is irrelevant to suppressing an oxidation stress or maintaining a skin homeostasis, and the like caused by physical damage or aging or the like. This is because the suppressing oxidation stress or the maintaining skin homeostasis and the like are not caused by mental stress, so the mechanism is totally different.

In addition, in the present specification, the mental stress does not mean a neurosis as referred to in a medical field, which means a maladapted status since a patient cannot adaptively adjust to psychological stress, but means a status of well adjusting to psychological stress but activating cortisone reductase (11β-hydroxysteroid dehydrogenase type 1) of keratinocytes in the epidermis regardless of the will of the parts concerned.

In the present specification, it will be understood that when an element such as a layer, film, region, or substrate is referred to as being “on” another element, it may be directly on the other element or intervening elements may also be present. In contrast, when an element is referred to as being “directly on” another element, there are no intervening elements present.

As used herein, when a definition is not otherwise provided, the term “combination” refers to mixing or copolymerization. In addition, “copolymerization” means block copolymerization or random copolymerization, and “copolymer” means block copolymer or random copolymer.

Hereinafter, a composition for inhibiting cortisone reductase according to an embodiment is described.

The composition for inhibiting cortisone reductase according to an embodiment includes a compound represented by Chemical Formula 1 as an active ingredient.

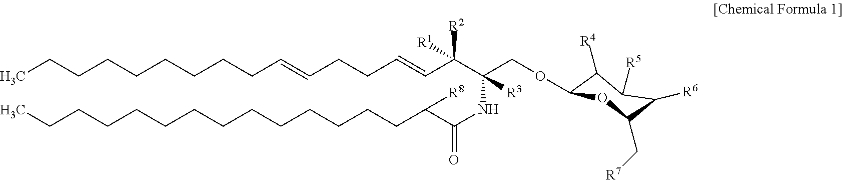

In Chemical Formula 1,

R¹ to R⁵ are independently a hydrogen atom or a hydroxy group, provided that at least one of R¹ to R⁵ is necessarily a hydroxy group.

For example, in Chemical Formula 1, R¹ and R³ may independently be a hydrogen atom, and R² and R⁴ to R⁵ may independently be a hydroxy group.

The compound represented by Chemical Formula 1 specifically inhibits the activity of cortisone reductase present in keratinocytes in the epidermis under a mental stress situation, so that the composition according to an embodiment may prevent deterioration of a skin barrier function even under the mental stress condition or rapidly improve the skin barrier function that is weakened by mental stress.

Specifically, under mental stress, since wound healing is delayed, and the skin barrier function is deteriorated, the firmness of the stratum corneum is also deteriorated, or recovery of the skin barrier function after the damage is delayed, which may be explained by two mechanisms. One mechanism is as follows; the metal stress activates an HPA (hypothalamus pituitary adrenal) axis to increase secretion of glucocorticoids (GC) in the blood through, and the glucocorticoids (GC) binds to GC receptors (GR) present in the epidermis and dermis, which are peripheral tissues, resulting in deteriorating the skin barrier function. The other mechanism is as follows, the mental stress activates cortisone reductase (11β-hydroxysteroid dehydrogenase 1) of keratinocytes in the epidermis, thereby increasing a concentration of cortisol (an activated GC form) and resulting in deteriorating the skin barrier function. The composition according to an embodiment may prevent the deterioration of the skin barrier function by inhibiting the activation of the cortisone reductase (11β-hydroxysteroid dehydrogenase 1) according to the second mechanism.

For example, the compound represented by Chemical Formula 1 may be a soybean extract. The compound represented by Chemical Formula 1 may be a rice extract, and this compound represented by Chemical Formula 1 derived from rice may also be used for a cosmetic composition but only serve to improve moisturizing properties, not playing a role in inhibiting the activation of cortisone reductase (11β-hydroxysteroid dehydrogenase 1) related to the mental stress. In other words, when the compound represented by Chemical Formula 1 is an extract derived from soybeans, the activation of the cortisone reductase (11β-hydroxysteroid dehydrogenase 1) may be most effectively inhibited. In addition, when the compound represented by Chemical Formula 1 is an extract from other materials besides the soybeans, the extract exhibits a completely different mechanism of enhancing the skin barrier from that of the soybean extract.

The cortisone reductase may be 11β-hydroxysteroid dehydrogenase type 1. The compound represented by Chemical Formula 1 extracted from soybeans and included in the composition according to an embodiment as an active ingredient may specifically act on the 11β-hydroxysteroid dehydrogenase type 1 and thus inhibit the activation of the cortisone reductase.

Accordingly, an embodiment provides a composition for inhibiting cortisone reductase including the compound represented by Chemical Formula 1 extracted from soybeans as an active ingredient and specifically, a composition for inhibiting activation of 11β-hydroxysteroid dehydrogenase type 1, which may include a pharmaceutically effective amount of the t compound represented by Chemical Formula 1 alone or along with at least one pharmaceutically acceptable carrier, excipient, or diluent.

The soybeans may include beans selected from soybeans, peas, and mung beans, germinated beans germinated from the beans, or a combination thereof. Specifically, the composition for inhibiting cortisone reductase according to an embodiment may include the compound represented by Chemical Formula 1 or a natural product containing the compound represented by Chemical Formula 1 and its extract as an active ingredient, and the compound represented by Chemical Formula 1 or the natural product containing and its extract may be obtained from beans such as soybeans, peas, and mung beans, germinated beans germinated from the beans, or a combination thereof. In addition, the extract of the natural product containing the compound represented by Chemical Formula 1 may be obtained by collecting an extract through cold precipitation at room temperature or warm precipitation of the natural product containing the compound represented by Chemical Formula 1 with 70% ethanol, completely concentrating it, dispersing it again in water, and fractionally collecting it again with at least one or two solvents selected from hexane, dichloromethane, chloroform, ethylacetate, butanol, ethanol, methanol, and water in the same amount. However, the extraction method is not limited thereto but may include all extraction methods of containing the compound represented by Chemical Formula 1 in a final product.

The compound represented by Chemical Formula 1 may be included in a concentration range of 0.01 μg/ml to 1000 μg/ml, in the composition. When the compound represented by Chemical Formula 1 is used as a cosmetic composition for inhibiting cortisone reductase, the compound represented by Chemical Formula 1 may be used at a concentration of greater than or equal to 0.01 μg/ml, greater than or equal to 0.1 μg/ml. The compound represented by Chemical Formula 1 may be used at a concentration of less than or equal to 1000 μg/ml, less than or equal to 100 μg/ml. When the compound represented by Chemical Formula 1 is used at a concentration of less than 0.01 μg/ml, there may be insignificant effects of proliferating epidermal keratinocytes and improving the skin battier function, but when the compound represented by Chemical Formula 1 is used at a concentration of greater than 1000 μg/ml, cytotoxicity may appear and thus harm the human body, which is not desirable.

In the above, “pharmaceutically effective amount” refers to an amount sufficient to allow the physiologically active ingredient to be administered to an animal or human to exhibit desired physiological or pharmacological activity. However, the effective amount of the pharmaceutical may vary according to the degrees of symptoms, ages, weights, health status, sexes, administration routes, and duration of treatment.

In addition, “pharmaceutically acceptable” refers to physiologically acceptable when administered to humans, and usually does not cause allergic reactions or similar reactions, such as gastrointestinal disorders or dizziness.

Examples of the carrier, excipient, and diluent may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oils. In addition, it may further include fillers, anti-coagulants, lubricants, wetting agents, fragrances, emulsifiers, and antiseptics.

For example, the composition may be a cosmetic composition.

In the present specification, “cosmetic” may refer to any material that may have a medical function in addition to the cosmetic function.

The formulation of the cosmetic composition is not particularly limited and may be appropriately selected as desired.

For example, the cosmetic composition may be formulated into formulations such as solutions, suspension liquids, emulsions, pastes, gels, creams, lotions, powders, soaps, surfactant-containing cleansings, oils, powder foundations, emulsion foundations, wax foundations, and sprays, but is not limited thereto. More specifically, it may be formulated into cosmetic compositions such as detergents, tonics, hair dressings, nourishing lotions, essences, serums, treatments, conditioners, shampoos, lotions, wools, or hair dyes, and the like, and may be formulated into basic cosmetics such as an oil-in-water (O/W) type, a water-in-oil (W/O), and the like. In addition, in the composition, in addition to the above-mentioned essential components in each formulation, other components may be appropriately selected and formulated without difficulty by a person of ordinary skill in the art according to types or use purposes of other external preparations. For example, ultraviolet blocking agents, hair conditioning agents, fragrances, and the like may be further included.

The cosmetic composition may include a cosmetically acceptable medium or base. These are all formulations suitable for topical applications. The cosmetic composition may be provided in the form of emulsions obtained by dispersing an oil phase in an aqueous phase, suspensions, microemulsions, microcapsules, microgranules, or ion-type (liposome) and/or non-ionized vesicle dispersing agents, or in the form of creams, skins, lotions, powders, ointments, sprays, or conceal sticks. These compositions may be prepared according to conventional methods in the art.

When the formulation of one aspect of the present disclosure is a solution or emulsion, a solvent, a solubilizer, or an emulsifier may be used as carrier components. For example, water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or fatty acid ester of sorbitan may be used.

If the formulation of one aspect of the present disclosure is a suspension, the carrier component may be a diluent of a liquid such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, tragacanth, and the like.

If the formulation of one aspect of the present disclosure is pastes, creams, or gels, the carrier component may be animal oil, vegetable oil, wax, paraffin, starch, tragacanth, cellulose derivatives, polyethylene glycol, silicone, bentonite, silica, talc, or zinc oxide.

If the formulation of one aspect of the present disclosure is powders or sprays, the carrier component may be lactose, talc, silica, aluminum hydroxide, calcium silicate, or polyamide powders. Particularly, in the case of sprays, a propellant such as a chlorofluorohydrocarbon, propane/butane, or dimethyl ether may be additionally included.

In an embodiment of one aspect of the present disclosure, the cosmetic composition may include thickeners. The thickeners included in the cosmetic composition of one aspect of the present disclosure may be methyl cellulose, carboxyl methyl cellulose, carboxyl methyl hydroxy guanine, hydroxy methyl cellulose, hydroxyethyl cellulose, a carboxyl vinyl polymer, polyquaternium, cetearyl alcohol, stearic acid, and carrageenan, preferably one or more of carboxyl methyl cellulose, a carboxyl vinyl polymer, and polyquaternium may be used, and more preferably a carboxyl vinyl polymer may be used.

In an embodiment of one aspect of the present disclosure, the cosmetic composition may include a variety of suitable bases and additives as needed, and the types and amounts of these components may be easily selected by the inventor. If necessary, it may include an acceptable additive, and may further include, for example, conventional ingredients such as antiseptics, pigments, additives, and the like.

The antiseptics may specifically be phenoxyethanol or 1,2-hexanediol, and the fragrances may be artificial fragrances.

In an embodiment of one aspect of the present disclosure, the cosmetic composition may include a composition selected from a water-soluble vitamin, an oil-soluble vitamin, a polymeric peptide, a polymeric polysaccharide, a sphingolipid, and a seaweed extract. Other ingredients that may be added include fats and oils humectants, emollients, surfactants, organic and inorganic pigments, organic powders, ultraviolet absorbers, antiseptics, fungicides, antioxidants, plant extracts, pH adjusters, alcohols, pigments, fragrances, blood circulation accelerators, coolants, anhidrotics, purified water, and the like.

In addition, the compounding components which may be added other than these are not limited thereto. Moreover, any component may be blended in the range which does not damage the purpose and effect of the invention.

Furthermore, the cosmetic composition according to an embodiment may be used not only as a pharmaceutical composition as described above, but also as a dietary supplement. For example, it may be easily used as main ingredients, auxiliary ingredients, food ingredients, food additives, functional foods, or beverages.

The “food” means a natural or processed product including one or more nutrients, and preferably means that it is ready to be eaten directly after a certain amount of processing. It includes all foods, food additives, functional foods, and beverages.

The foods to which the food composition can be added may include, for example, various foods, beverages, gums, teas, vitamin composites, and functional foods. In addition, the foods may include special nutritional products (e.g., formulas, baby food, etc.), processed meat products, fish products, tofu, jellies, noodles (e.g. ramen noodles, etc.), breads, dietary supplements, seasoned foods (e.g., soy sauce, soybean paste, red pepper paste, mixed soy sauce, etc.), sauces, sweets (e.g. snacks), candy, chocolate, gum, ice cream, dairy products (e.g. fermented milk, cheese, etc.), other processed foods, kimchi, pickles (various kimchi, pickles, etc.), beverages (e.g., fruit beverages, vegetable beverages, soy milk, fermented beverages, etc.), natural seasonings (e.g., ramen soup, etc.), but are not limited thereto. The foods, beverages, or food additives may be prepared by conventional preparing methods.

In addition, “functional foods” or “health functional foods” refers to a food group that has added values to foods by using physical, biochemical, or biotechnological techniques to act and express functions of foods for specific purposes, or foods that are processed and designed to fully express the body's regulatory functions, such as defense rhythm control of food compositions, disease prevention, and recovery of living bodies. It may specifically be a health functional food. The functional food may include acceptable food auxiliary additives, and may further include suitable carriers, excipients, and diluents commonly used in the manufacture of functional foods.

The types of dietary supplements are not limited thereto, but may be in a form of powders, granules, tablets, capsules, or beverages.

According to another embodiment, provided is a method of inhibiting cortisone reductase by applying a composition including an effective amount of the compound represented by Chemical Formula 1 as an active ingredient, to the skin.

Advantages and features of one aspect of the present disclosure and methods for achieving them will be apparent with reference to the examples described in detail below. One aspect of the present disclosure will be described in detail with reference to examples. However, these examples are specifically provided for describing one aspect of the present disclosure, and the range of one aspect of the present disclosure is not limited to these examples.

### EXAMPLES

### Experimental Example 1: Cortisone Reductase Activity Inhibitory Effect

Normal human epidermal keratinocytes (NHEK) were cultured in a 6-well plate incubator. Specifically, the normal human epidermal keratinocytes (NHEK) were cultured for 4 days by replacing it with phosphate-buffered saline (PBS) after 24 hours to irradiate UVB (25 mJ/cm²) and adding cortisone (10 μg/ml) and a compound represented by Chemical Formula 1-1 (glucocerebrosides (soy, 98%), Avanti Polar Lipids, Inc.) to NHEK culture media.

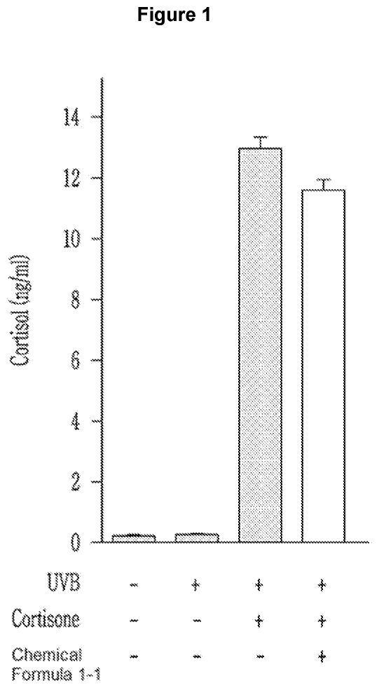

Subsequently, the cell culture solution was collected to measure a cortisol concentration in an ELISA method, and the results are shown in FIG. 1. Referring to FIG. 1, the cortisol concentration in a well to which the cortisone was added after the ultraviolet ray irradiation (UVB 25 mJ/cm²) greatly increased, compared with a well to which the cortisone was added before the ultraviolet ray irradiation (UVB 25 mJ/cm²), but when the compound represented by Chemical Formula 1-1 (10 μg/ml) was also added to the wells, the cortisol concentration greatly did not increase. Accordingly, a composition including the compound represented by Chemical Formula 1-1 as an active ingredient according to an embodiment turned out to inhibit activity of 11β-hydroxysteroid dehydrogenase type 1 that is cortisone reductase.

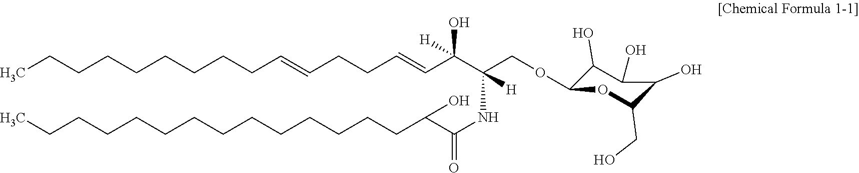

### Experimental Example 2: Cortisone Reductase Activity Inhibitory Effect

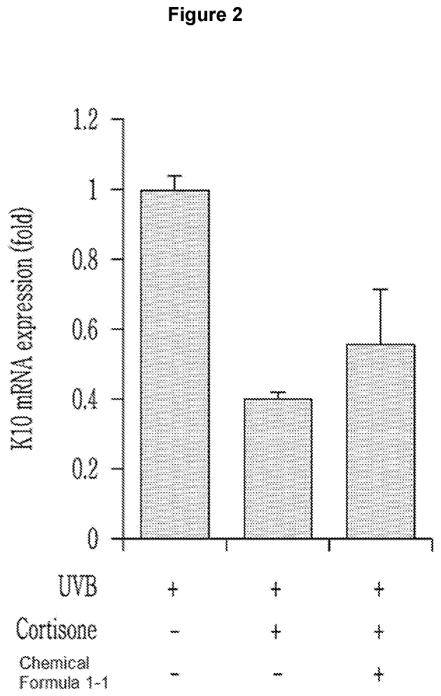

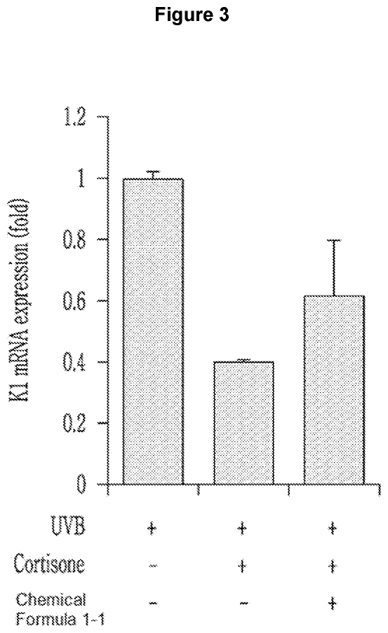

In addition, the normal human epidermal keratinocytes (NHEK) were treated with TRIZOL to separate RNA, which was used to synthesize cDNA through RT-PCR (reverse transcriptional polymerase chain reaction). The synthesized cDNA was used to perform TAQMAN® real-time PCR and measure gene expression amounts of keratinocyte differentiation markers of KRT1 and KRT10, and the results are shown in FIGS. 2 and 3. Referring to FIGS. 2 and 3, in the well to which the compound represented by Chemical Formula 1 (10 μg/ml) was added, the gene expression of the keratinocyte differentiation markers (KRT10, KRT1) inhibited by ultraviolet (UVB) rays in the keratinocytes was increased. In other words, the gene expression of the keratinocyte differentiation markers inhibited by the ultraviolet (UVB) rays in the keratinocytes was restored by the compound represented by Chemical Formula 1, and accordingly, the compound represented by Chemical Formula 1 inhibited the activity of the cortisone reductase to reduce a cortisol concentration, resulting in restoring the gene expression of the keratinocyte differentiation markers.

Although the preferred embodiments of one aspect of the present disclosure have been described in detail, the scope of one aspect of the present disclosure is not limited thereto, and various modifications and improvements by those skilled in the art using the basic concept of one aspect of the present disclosure defined in the following claims are also within the scope of the invention.

1. A method of inhibiting cortisone reductase by applying an effective amount of a composition to skin of a subject,
wherein, the composition comprises
a compound of the following Chemical Formula 1 as an active ingredient: 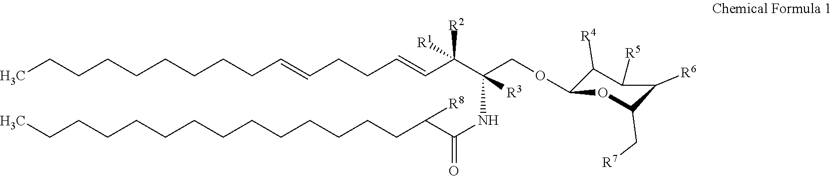
wherein, in Chemical Formula 1,
R¹ to R⁸ are each independently a hydrogen atom or a hydroxy group, provided that at least one of R¹ to R⁸ is a hydroxy group.

2. The method of claim 1, wherein
R¹ and R³ are each independently a hydrogen atom, and
R² and R⁴ to R⁸ are each independently a hydroxy group.

3. The method of claim 1, wherein the compound represented by Chemical Formula 1 is a soybean extract.

4. The method of claim 1, wherein the composition inhibits cortisone reductase,
wherein, the cortisone reductase is 11β-hydroxysteroid dehydrogenase type 1.

5. The method of claim 1, wherein the compound of Chemical Formula 1 is included in a concentration range of 0.01 μg/ml to 1,000 μg/ml in the composition.

6. The method of claim 1, wherein the composition is a cosmetic composition.
